(19) United States
(12) Patent Application Publication
(10) Pub. No.: US 2017/0209117 A1
(43) Pub. Date: Jul. 27, 2017

(54) SYSTEM AND METHOD FOR MEASUREMENT OF LONGITUDINAL AND CIRCUMFERENTIAL WAVE SPEEDS IN CYLINDRICAL VESSELS

(71) Applicant: MAYO FOUNDATION FOR MEDICAL EDUCATION AND RESEARCH, Rochester, MN (US)

(72) Inventors: Matthew W. Urban, Rochester, MN (US); Shigao Chen, Rochester, MN (US); James F. Greenleaf, Rochester, MN (US)

(21) Appl. No.: 15/329,535

(22) PCT Filed: Jul. 29, 2015
(86) PCT No.: PCT/US2015/042718

§ 371 Date: Jan. 26, 2017

Related U.S. Application Data
(60) Provisional application No. 62/030,109, filed on Jul. 29, 2014.

(51) Int. Cl. A61B 8/12; A61B 5/02; A61B 8/08; A61B 5/00

(52) U.S. Cl. CPC A61B 8/12; A61B 5/0048; A61B 5/02007; A61B 5/7235; A61B 8/0891; A61B 8/485

(57) ABSTRACT

Systems and methods are provided for measuring and isolating circumferential wave speed within a vessel wall of a substantially cylindrical vessel. The methods include measuring a motion at a first location and a second location opposite the first location and isolating the circumferential wave speed. The methods also include generating a report using the longitudinal or circumferential wave speeds.

### CROSS-REFERENCE TO RELATED APPLICATIONS

This application claims priority to U.S. Provisional Patent Application No. 62/030,109, filed Jul. 29, 2014, the entire contents of which are incorporated herein in their entirety by reference.

### STATEMENT REGARDING FEDERALLY SPONSORED RESEARCH

This invention was made with government support under EB002640 awarded by the National Institutes of Health. The government has certain rights in the invention.

### BACKGROUND OF THE INVENTION

The present disclosure relates to systems and methods for measuring and isolating wave speed within a vessel wall. More particularly, the present disclosure relates to systems and methods for measuring and isolating wave speed within a vessel wall of a generally cylindrical vessel with an imaging system, such as an ultrasound imaging system.

Increased arterial stiffness is associated with increased risk of cardiovascular events. The measurement of the pulse wave velocity (PWV), which is the speed at which the pressure pulse generated from the blood ejection from the left ventricle of the heart travels through the arterial tree, can be used to estimate arterial stiffness. For instance, carotid-femoral PWV (cfPWV), also known as aortic PWV, is considered a “gold standard” measure of arterial stiffness. It is measured by applanation tonometry of the carotid and femoral arteries to obtain pressure waveforms and transit time, and over the surface body measurements are used to estimate the distance the wave has traveled. The cfPWV is related to the Young's modulus, E, of the arterial wall by the Moens-Korteweg equation, cfPWV=\sqrt{Eh/2Rρ}, where h is the wall thickness, R is the inner radius, and ρ is the density of the blood. The cfPWV is a global measurement or a measurement over a very long segment of the arterial tree. Measuring over a long segment can introduce large bias errors because of inexact knowledge of the true length of the arterial segment over which the wave has traveled.

A more local measurement of PWV can be made using a method call pulse wave imaging (PWI). This method measures the motion of the arterial or heart wall in order to track the wave propagation through the tissue. This method has been utilized in the aortas of mice with and without aneurysms, and in human aortas and carotid arteries. This method has the advantage of assessing the PWV over a short segment (1-9 cm) compared to the longer segment used clinically in cfPWV, which is on the order of tens of centimeters. One limitation of PWI is that only one measurement can be made for each cardiac cycle, so the spatial resolution is higher than traditional cfPWV methods, but the temporal resolution is no better.

Acoustic radiation force has been utilized for perturbing arterial vessels for material characterization. Acoustic Radiation Force Impulse (ARFI) imaging which focuses high intensity ultrasound to displace the tissue has been used to explore localized plaque formations in arterial vessels. Analysis of the displacement amplitude and time course was used in swine models to investigate the ability to localize and classify atherosclerotic plaques. Another method called Supersonic Shear Imaging (SSI) uses focused ultrasound to make propagating shear waves in tissue and has been used to create high frequency propagating waves in human carotid arteries to measure the material properties through the cardiac cycle.

Previous work has used a method called Shearwave Dispersion Ultrasound Vibrometry (SDUV), which uses ultrasound to generate and measure shear waves in soft tissues and analyze the shear wave velocity dispersion to extract viscoelastic material properties. It used acoustic radiation force in tubes and ex vivo arteries to produce high frequency waves (100-500 Hz) in the wall of the tube or vessel to investigate the material properties, similar to the application of the SDUV method in large organs.

The artery's material properties are known to be anisotropic. This has been established by many studies that have examined ex vivo arteries after being cut using biaxial testing to evaluate the behavior of the artery in the longitudinal and circumferential directions. An in vivo study in rats demonstrated biaxial testing of carotid artery passive and active stiffness and the variation with age of the rats. Both the biaxial passive and active stiffness increased in rats evaluated at 6 and 23 months.

Therefore, it would be desirable to have a system and method for measuring and reporting material properties of a vessel in vivo.

### SUMMARY OF THE INVENTION

The present disclosure overcomes the aforementioned drawbacks by providing systems and methods for measuring wave speeds in substantially cylindrical vessels. Systems and methods described herein are suitable for measuring and isolating a wave speed within a vessel wall of a substantially cylindrical vessel by isolating the longitudinal and circumferential wave speeds in the substantially cylindrical vessel to determine material properties of the vessel using the isolated wave speeds.

In accordance with the present disclosure, the method of measuring and isolating a wave speed within a vessel wall of a substantially cylindrical vessel using an ultrasound imaging system, the vessel having a longitudinal axis and a circumference that is orthogonal to the longitudinal axis, the method includes measuring, using a first transducer of the ultrasound imaging system, a motion of the vessel wall at a first location on the circumference, the vessel wall experiencing a propagating longitudinal wave along the longitudinal axis and a propagating circumferential wave about the circumference. The method also includes measuring, using the first transducer or a second transducer of the ultrasound imaging system, a motion of the vessel wall at a second location on the circumference. The first and second location can be positioned substantially opposite one another on the circumference. The method also includes isolating a circumferential wave speed of the propagating circumferential wave by computationally analyzing, using a processor of the ultrasound imaging system, a difference between the motion of the vessel wall at the first location and the motion of the vessel wall at the second location. The method also includes generating a report indicating material properties of the vessel using the longitudinal or circumferential wave speeds.

In accordance with the present disclosure, the method of measuring and isolating a wave speed within a vessel wall of a substantially cylindrical vessel using a motion imaging system, the vessel having a longitudinal axis and a circumference that is orthogonal to the longitudinal axis, the method includes inducing a propagating longitudinal wave within the vessel wall along the longitudinal axis and a propagating circumferential wave within the vessel wall about the circumference. The method also includes measuring, using a first motion detector of the motion imaging system, a motion of the vessel wall at a first location on the circumference. The method also includes measuring, using the first motion detector or a second motion detector of the motion imaging system, a motion of the vessel wall at a second location on the circumference, the first and second location are positioned substantially opposite one another on the circumference. The method also includes isolating a circumferential wave speed of the propagating circumferential wave by Fourier transforming a difference between the motion of the vessel wall at the first location and the motion of the vessel wall at the second location. The method also includes generating a report using the longitudinal or circumferential wave speeds.

The foregoing and other aspects and advantages of the invention will appear from the following description. In the description, reference is made to the accompanying drawings that form a part hereof, and in which there is shown by way of illustration a preferred embodiment of the invention. Such embodiment does not necessarily represent the full scope of the invention, however, and reference is made therefore to the claims and herein for interpreting the scope of the invention.

### BRIEF DESCRIPTION OF THE DRAWINGS

FIG. 1 is a schematic of an ultrasound imaging system, in accordance with the present disclosure.

FIG. 2 is a flow chart setting forth steps of an example of a method in accordance with the present disclosure.

FIGS. 3A and 3B are schematic views of an example arrangement of ultrasound transducers suitable for measuring and isolating circumferential waves in a vessel, bodily lumen, or other tubular structure, in accordance with the present disclosure.

FIG. 4 is plot showing motion of a tube as measured by the push/detect transducer at points P1 and P2 of FIG. 3B.

FIG. 5 is a plot showing motion of a tube as measured by the detect transducer at points P3 and P4 of FIG. 3B.

FIG. 6 is a plot of the sum of wall motion of two opposite walls of an artery at 40 mm Hg.

FIG. 7 is a plot of the difference of wall motion of two opposite walls of an artery at 40 mm Hg.

FIG. 8 is a plot of the normalized fast Fourier transform near x=0 mm for the sum and difference shown in FIGS. 6 and 7, respectively.

FIG. 9 is a plot of the circumferential wave speeds versus the longitudinal wave speeds for two tubes.

FIG. 10 is a plot of the longitudinal and circumferential wave speeds in an ex vivo artery versus pressure over a transmural pressure range of 20-200 mm Hg.

FIG. 11 is a schematic illustration of using unfocused ultrasound to simultaneously excite longitudinal and circumferential waves in both the near-side wall of a vessel and the far-side wall of a vessel.

FIG. 12 is a schematic illustration of using focused ultrasound beams to excite longitudinal and circumferential waves in both the near-side wall of a vessel and the far-side wall of a vessel by focusing and/or steering focused ultrasound to two different depths.

FIG. 13 is a schematic illustration of using a single focused ultrasound beam to simultaneously excite longitudinal and circumferential waves in both the near-side wall of a vessel and the far-side wall of a vessel, where the focused ultrasound beam is designed to have a focal region that is long enough to encompass both sides of the vessel.

FIG. 14 is a schematic illustration of using two unfocused ultrasound beams positioned at different lateral locations along the longitudinal axis of a vessel to simultaneously excite longitudinal and circumferential waves in both the near-side wall of a vessel and the far-side wall of a vessel.

### DETAILED DESCRIPTION OF THE INVENTION

This disclosure provides methods of measuring and isolating a wave speed within a wall of a vessel, such as a bodily lumen or other tubular structure, using a motion imaging system. As an example, a bodily lumen can include a blood vessel, the esophagus, a section of the gastrointestinal tract, or the like.

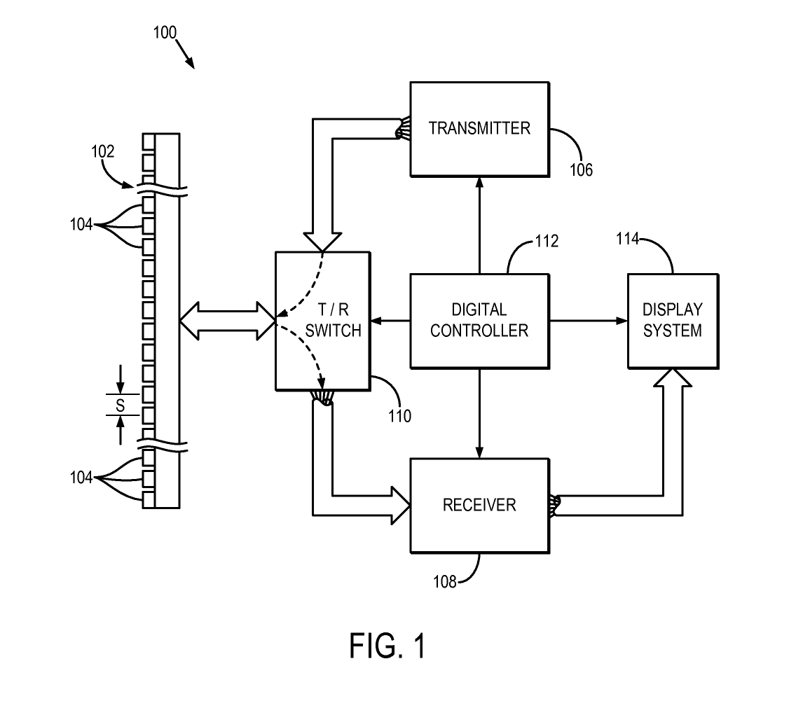

Referring particularly to FIG. 1, an example of an ultrasound imaging system 100 (an example of a motion imaging system) includes a transducer array 102 that includes a plurality of separately driven transducer elements 104. In some configurations, the transducer array 102 may include a linear array transducer.

When energized by a transmitter 106, each transducer element 104 produces a burst of ultrasonic energy. The ultrasonic energy reflected back to the transducer array 102 from the object or subject under study is converted to an electrical signal by each transducer element 104 and applied separately to a receiver 108 through a set of switches 110. The transmitter 106, receiver 108, and switches 110 are operated under the control of a digital controller 112 responsive to commands input by a user. A complete scan is performed by acquiring a series of echo signals in which the switches 110 are set to their transmit position, thereby directing the transmitter 106 to be turned on momentarily to energize each transducer element 104. The switches 110 are then set to their receive position and the subsequent echo signals produced by each transducer element 104 are measured and applied to the receiver 108. The separate echo signals from each transducer element 104 are combined in the receiver 108 to produce a single echo signal that is employed to produce a line in an image, for example, on a display system 114.

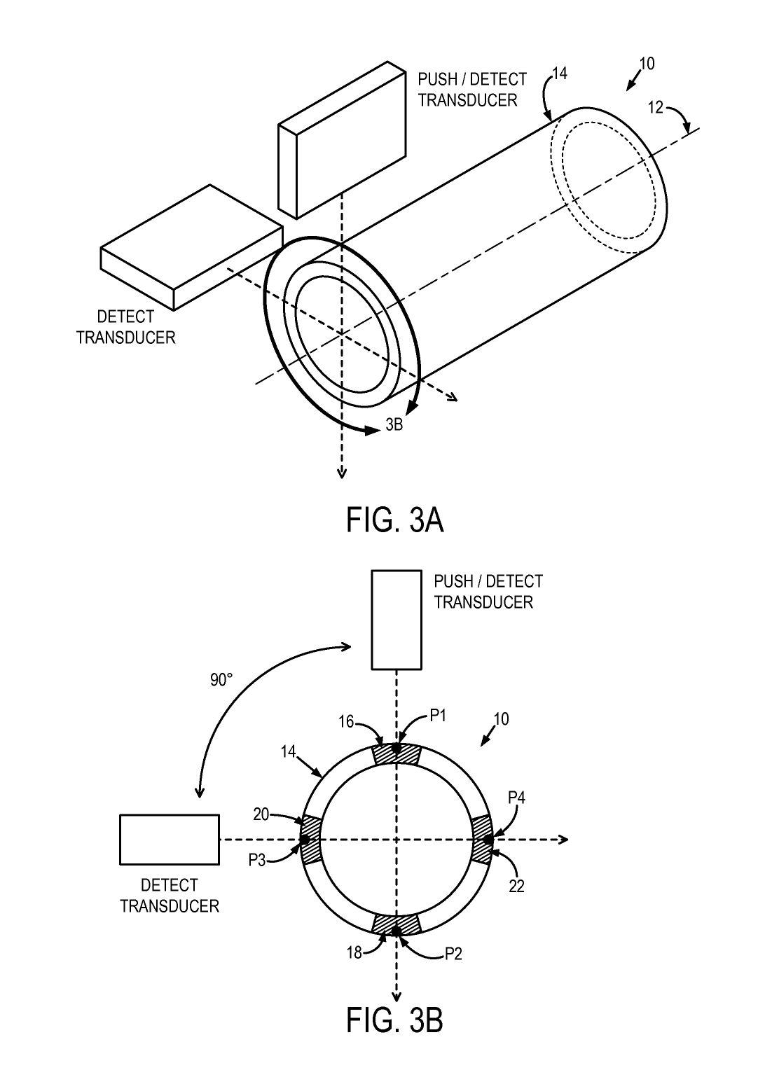

The present disclosure recognizes that motion imaging using a system such as described with respect to FIG. 1 offers the ability to measure and isolate circumferential wave speeds. This disclosure provides systems and methods for measuring and isolating a wave speed within a vessel wall of a substantially cylindrical vessel. The vessel includes a longitudinal axis and a circumference that is orthogonal to the longitudinal axis. For instance, as shown in FIGS. 3A and 3B, the vessel 10 has a longitudinal axis 12 and a circumference 14 that is orthogonal to the longitudinal axis. The circumference includes a first location, such as point P1, in a first region 16; a second location, such as point P2, in a second region 18; a third location, such as point P3, in a third region 20; and a fourth location, such as point P4, in a third region 22. In general, the first region 16 is proximal to the ultrasound transducer used to induce longitudinal and circumferential waves in the vessel 10 and the second region 18 is distal to the ultrasound transducer used to induce longitudinal and circumferential waves in the vessel 10. For instance, the first region 16 can be on the near-side of the vessel 10 and the second region can be on the far-side of the vessel 10. In some instances, the first region 16 and second region 18 can be positioned substantially opposite one another on the circumference 14. In some instances, the third region 20 and fourth region 22 can be positioned substantially opposite one another on the circumference 14 and at an angle of about 90° relative to the first location 16 and the second location 18.

The methods may include one or more of the following: inducing a propagating longitudinal wave within a vessel wall along a longitudinal axis; inducing a propagating circumferential wave within a vessel wall about a circumference that is orthogonal to the longitudinal axis; measuring a motion of a vessel wall at a first location on the circumference; measuring a motion of the vessel wall at a second location on the circumference, the first location and second location are positioned substantially opposite one another on the circumference; isolating the circumferential wave speed of the propagating circumferential wave; isolating the longitudinal wave speed of the propagating longitudinal wave; and generating a report using the circumferential wave speed or the longitudinal wave speed.

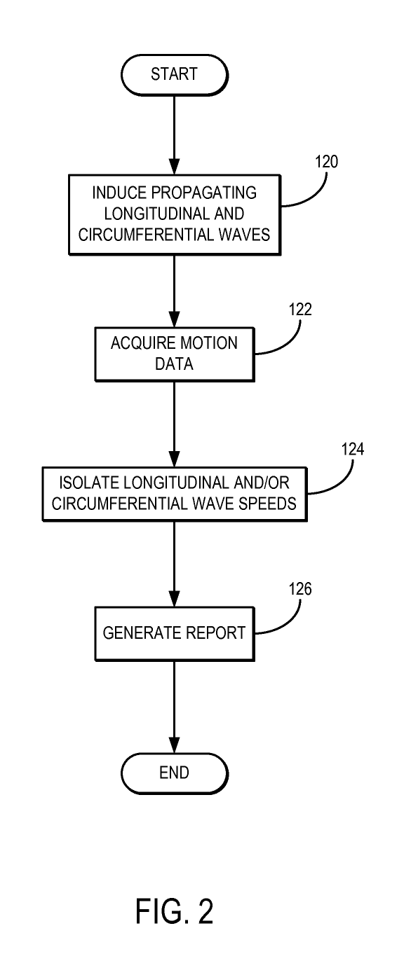

In particular, referring to FIG. 2, a flow chart is provided setting forth examples of steps of a method that may be performed in accordance with the present disclosure for measuring and isolating a wave speed within a vessel wall of a vessel, which may be a substantially cylindrical vessel.

At process block 120, a propagating longitudinal wave within the vessel wall along the longitudinal axis and a propagating circumferential wave within the vessel wall about the circumference are optionally induced. In certain aspects, the methods do not require inducing the propagating waves, because the vessel may already contain propagating waves. For example, in some instances intrinsic physiological motion will generate longitudinal and circumferential waves in a vessel or other bodily vessel. As one example, pulsatile blood flow can induce longitudinal and circumferential waves in the wall of a blood vessel. As another example, swallowing can induce longitudinal and circumferential waves in the wall of the esophagus. In certain configurations, the propagating waves may also be induced by a force inducing element of the ultrasound imaging system, such as by generating ultrasound with a transducer.

In certain aspects, the propagating waves are induced by delivering a force to the vessel. In certain aspects, the force that is delivered is a force that induces shear strain.

In certain aspects, the propagating waves are induced by an acoustic radiation force, an external vibration, or a combination thereof. Acoustic radiation force and external vibration each has their advantages in terms of frequency bandwidth, ease of application, signal-to-noise ratio, and modal vibration, among others. Acoustic radiation force can provide a targeted, high bandwidth wave, but can be limited in motion amplitude. External vibration can provide large motion and does not require any high power pulses from an ultrasound transducer for generation of the wave, but does require a component that is external to the transducer.

The acoustic radiation force can be focused or unfocused. The radiation force can also be generated using one or more beams. The acoustic radiation force can be distributed at different locations along the vessel wall. In aspects where the acoustic radiation force is focused, the force can be applied at a force push location on the vessel wall. The force push location can be located on the circumference and can be located at the first location, second location, third location, or fourth location. In certain aspects, multiple beams of acoustic radiation force can be applied at multiple locations along the longitudinal direction.

The force can be applied with any waveform that a person having ordinary skill in the art would recognize as suitable for imaging applications, such as a pure sinusoidal waveform, a chirped waveform, a waveform where the frequency varies over time, combinations thereof, and the like. The motion imaging system can further comprise a pulse generator for designing and executing waveforms.

The force inducing element can include a transducer of an ultrasound imaging system, a linear actuator, such as a speaker, an electromechanical shaker, or a source of vibrations, or a combination thereof. In certain aspects, the force inducing element is located within the first or second transducer.

In some aspects, longitudinal and circumferential waves are induced in a vessel wall using one or more unfocused ultrasound push beams that are optimized to produce and isolate circumferential waves. To isolate the circumferential wave, it is important to cancel out the bending wave or anti-symmetric motion. To achieve this, the motion induced in the vessel has to be similar on the near-side and far-side vessel walls in order for the subtraction to leave only the circumferential motion. Preferably, both walls would be stimulated with a similar force to produce an anti-symmetric amplitude that is equal in each wall.

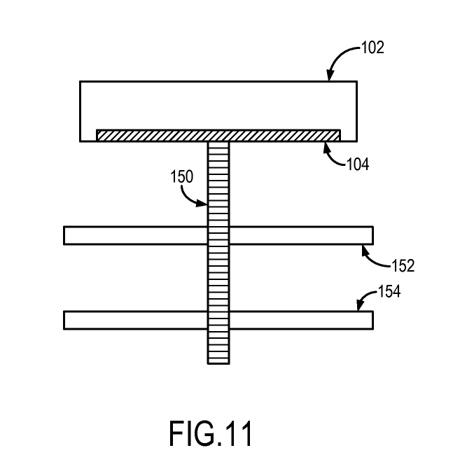

Referring to FIG. 11, an unfocused push beam 150 can be employed by turning on several contiguous transducer elements 104 in the ultrasound transducer 102. This type of excitation produces a relatively uniform push beam 150 that has a large depth-of-field. Because of this large depth-of-field, using an unfocused push beam 150 results in the force on the near-side vessel wall 152 to be similar to the force on the far-side vessel wall 154. As a result, the motion in the near-side and far-side vessel walls 152, 154 can be subtracted to examine the circumferential wave propagating in the vessel.

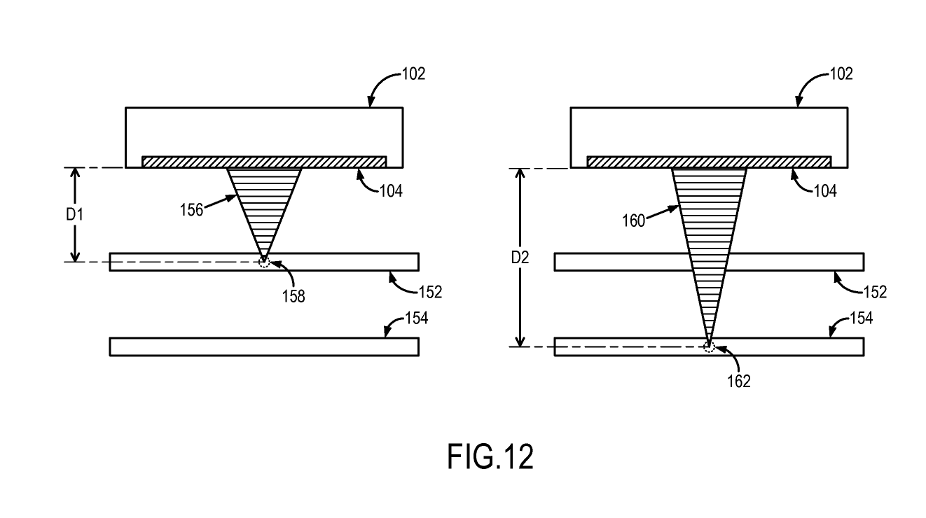

In some instances an unfocused push beam might not be strong enough to generate similar motion in both the near-side and far-side vessel walls. Thus, as illustrated in FIG. 12, in some aspects, longitudinal and circumferential waves are induced in a vessel wall using two focused push beams: one focused push beam 156 focused at a focal region 158 located in the near-side wall 152 and one focused push beam 160 focused at a focal region 162 located in the far-side vessel wall 154. This excitation scheme can be referred to as a “dual-depth focused beam” method. As one example, focused push beam 156 could be used to excite a wave preferentially in the near-side vessel wall 152 wall by electronically focusing the push beam 156 at a focal region 158 located in the near-side vessel wall 152 at depth D1. The motion data from this excitation can be recorded as M1. Then, focused beam 160 could be electronically focused in the focal region 162 located in the far-side vessel wall 154 at depth D2. The motion data from this excitation can be recorded as M2. The motion data from the near-side vessel wall 152 (M1) and the motion data from the far-side vessel wall 154 (M2) can then be subtracted to isolate the circumferential wave, or the same data sets could be added to isolate the longitudinal wave.

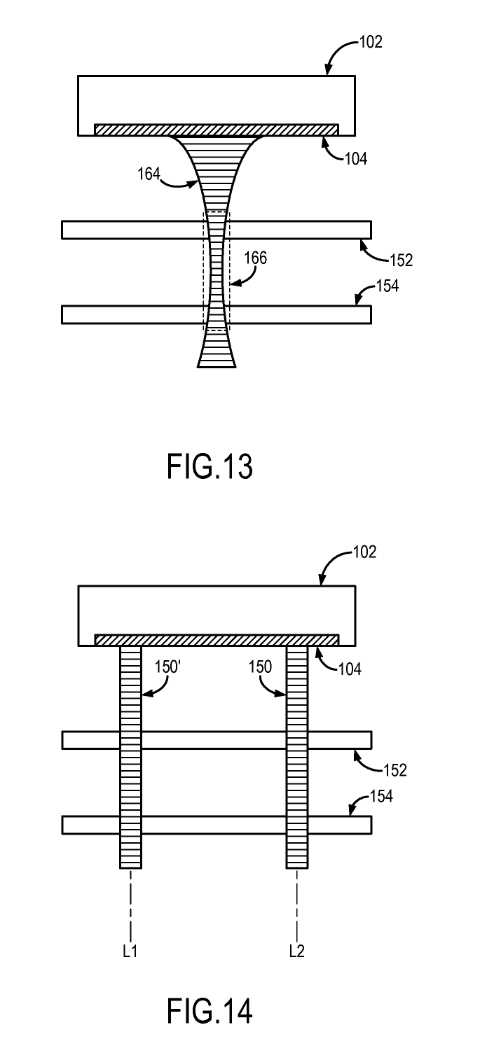

In some other embodiments, a single focused ultrasound beam can be used to simultaneously excite the near-side vessel wall 152 and the far-side vessel wall 154, as illustrated in FIG. 13. In this example, the focused ultrasound beam 164 is designed to have a focal region 166 that is long enough to simultaneously excite both the near-side vessel wall 152 and the far-side vessel wall 154.

For redundancy and robustness, any of the different push schemes (e.g., unfocused beam, single focused beam, dual-depth focused beam methods) can be implemented at different lateral locations in a simultaneous fashion. An example of this is illustrated in FIG. 14, where a first unfocused push beam 150 is directed at a first lateral location L1 while a second unfocused push beam 150′ is directed at a second lateral location L2. The motion induced by the push beams in this manner would be recorded and could be directionally filtered to separate waves that are moving from left-to-right along the longitudinal direction and waves that are moving right-to-left along the longitudinal direction. Addition and subtraction of the motion can also be carried out to isolate the longitudinal or circumferential waves, respectively.

Referring again to FIG. 2, at process block 122, motion data is acquired. Specifically, a motion imaging system, such as the ultrasound imaging system described with respect to FIG. 1, can be used to measure a motion of the vessel wall. In certain aspects, the motion imaging system may comprise an ultrasound imaging system, a magnetic resonance imaging system, an optical imaging system, or a combination thereof. The motion imaging system can comprise a plurality of motion detectors (for example, transducers).

In certain aspects, the methods comprise measuring a motion of the vessel wall at a first location on the circumference. Measuring the motion at the first location can be done using a first motion sensor of a motion imaging system or a first transducer of an ultrasound imaging system.

In certain aspects, the methods comprise measuring a motion of the vessel wall at a second location on the circumference. Measuring the motion on the second location can be done using the first motion sensor or the first transducer. Alternatively, measuring the motion on the second location can be done using a second motion sensor of the motion imaging system of a second transducer of the ultrasound imaging system. The methods may further comprise measuring a motion of the vessel wall at additional locations on the vessel, on the circumference or elsewhere along the longitudinal axis.

It should be appreciated that motion data should be acquired on a timescale that allows sufficient sampling of the propagating waves. In other words, the motion data should be acquired on a timescale that precludes undersampling of the propagating waves.

In certain aspects, the methods comprise measuring a motion of the vessel wall at a third and fourth location on the circumference.

In certain aspects, the motion of the vessel is measured over no more than about 10 cm of length of the vessel along the longitudinal direction.

The methods may further comprise selecting the first location and second location based on a geometry of the vessel. In certain aspects, the geometry of the vessel is measured using an imaging system, such as an ultrasound imaging system, a magnetic resonance imaging system, an optical imaging system, or a combination thereof. In certain aspects, the geometry may be measured, estimated, calculated, or the like.

At process block 124, longitudinal and/or circumferential wave speeds are isolated.

In certain aspects, the methods comprise isolating a circumferential wave speed of the propagating circumferential wave. Isolating the circumferential wave speed may comprise computationally analyzing or Fourier transforming a difference between the motion of the vessel wall at the first location and the motion of the vessel wall at the second location.

In certain aspects, the methods comprise isolating a longitudinal wave speed of the propagating longitudinal wave. Isolating the longitudinal wave speed may comprise using a time of flight based technique, using a Radon transform based technique, or using a Fourier transform based technique. In certain aspects, isolating the longitudinal wave speed may comprise Fourier transforming the sum of the motion of the vessel wall at the first location and the motion of the vessel wall at the second location.

In certain aspects, the methods comprise determining a material or mechanical property of the vessel using the circumferential wave speed or the longitudinal wave speed. The material properties can include density, elasticity, viscosity, stiffness, anisotropy, and the like.

In certain aspects, the methods comprise determining an anisotropy of the vessel using the circumferential wave speed or the longitudinal wave speed.

At process block 126, a report may be generated. The report may use the circumferential wave speed, the longitudinal wave speed, a material property of the vessel, or a combination thereof. It should be appreciated that use in the report does not need to be direct use in the report, and can include use in a computation whose result is provided in the report, use directly in the report, use in making a determination, prognostication, or categorization that is provided in the report, and the like.

In certain aspects, the substantially cylindrical vessel is a synthetic tube, a blood vessel, an esophagus, a section of the gastrointestinal tract, or the like. In certain aspects, the substantially cylindrical vessel is suspended within an external medium, such as human tissue, a gel, or the like. In certain aspects, the substantially cylindrical vessel can contain a gas, such as a pressurized gas, or a fluid or solid medium, such as blood, water, saline, a gel, or the like, within the vessel.

In certain aspects, the transducers may be linear array transducers. In certain aspects, measuring at a location may comprise measuring over a longitudinal section of the vessel wall that includes the location. In certain aspects, measuring at a location may comprise measuring over a circumferential section of the circumference.

In certain aspects, the methods further comprise confirming the presence of the propagating circumferential wave of the propagating longitudinal wave. Confirming can include measuring, using the motion imaging system, a motion of the vessel wall at the third location and a motion of the vessel wall at the fourth location on the circumference. The motion of the vessel wall at the first location and the motion of the vessel wall at the second location can be measured by the first motion detector of the motion imaging system or the first transducer of the ultrasound imaging system. The motion of the vessel wall at the third location and the motion of the vessel wall at the fourth location can be measured by the second motion detector of the motion imaging system or the second transducer of the ultrasound imaging system.

In certain aspects, the methods further comprise measuring a phase velocity of the propagating waves. The phase velocity may be measured using a Fourier transform based technique.

An external vibration source can be applied to skin to induce motion in the underlying tissue including the arterial wall. The external vibration can be a pure sinusoidal tone or a simultaneous combination of sinusoidal tones, or a more complicated signal such as a chirp, a signal that changes frequency through time. The actuator could be a small speaker or some other vibrating source such as an electromechanical shaker. The arterial wave motion can be assessed using ultrasound-based techniques using focused or unfocused beams at a sufficient frame rate to capture the frequency content of the external vibration. Analysis similar to that detailed above could be used. Additionally, data analysis using phase-based techniques utilizing Fourier transforms and moving windows through time can be used to measure the phase at a given frequency or over a specified bandwidth. A two-dimensional Fourier transform of the spatiotemporal data can be used to directionally filter the data as well as analyze wave velocities.

### EXAMPLES

Acoustic radiation force was used to generate propagating waves in the wall of rubber tubes and an excised porcine carotid artery and measured the wave motion using compounded plane wave imaging. To study the tubes, two Verasonics systems equipped with linear array transducers (L7-4, Philips Healthcare, Andover, Mass.) were used.

The transducers were placed at 90° with respect to each other to obtain different views along the tube wall (FIGS. 3A and 3B). One transducer applied a radiation force push and then both systems were used to detect the wall motion of the tube. One system was used for the artery experiment. The group velocity of the longitudinal wave, c₁, was measured along the length of the vessel/tube. The motion near the radiation force push location was analyzed to measure the speed of the circumferential wave. A Fourier transform was taken and the frequency of the peak motion was extracted. This frequency, f_{c}, is inversely proportional to the transit time needed for the wave to travel around the circumference of the cylinder such that the circumferential speed is c_{c}=πDf_{c}, where D is the outer diameter of the cylinder. The transmural pressure was varied over ranges of 10-30 mmHg and 20-200 mmHg for the tubes and artery, respectively. The transmural pressure was varied because the artery's moduli are nonlinear. At low pressures, the moduli increase linearly with pressure, and as pressure continues to rise, the moduli increase at different rates so pressure is important to note during measurements.

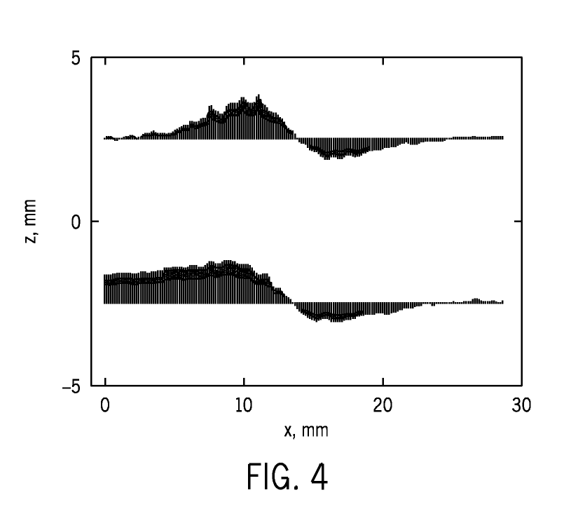

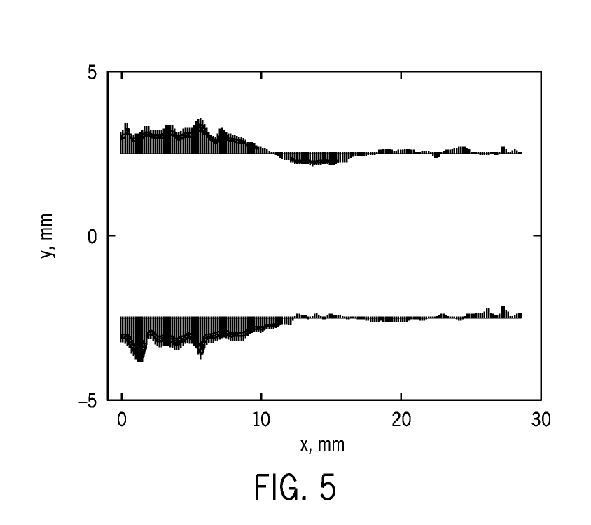

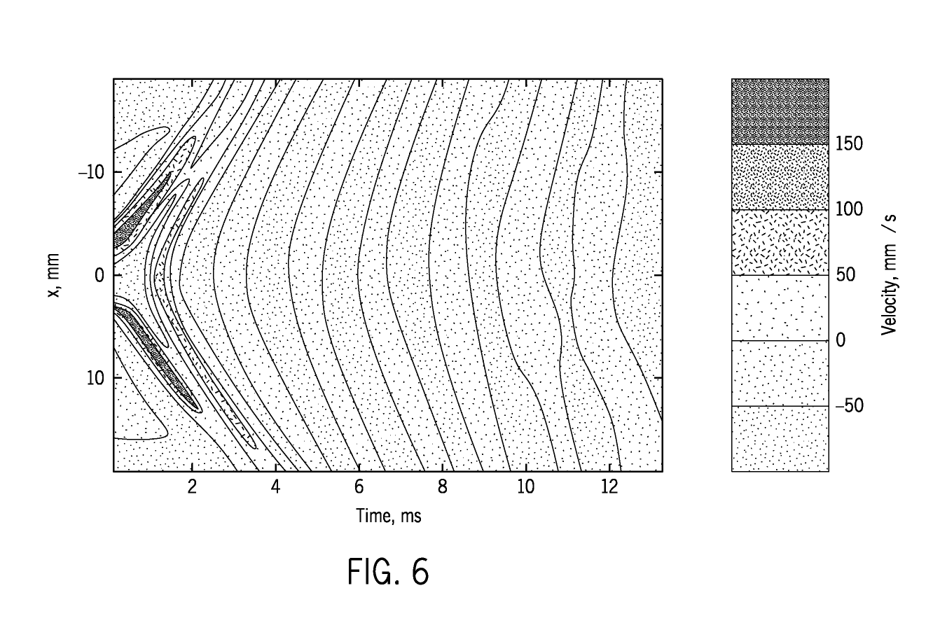

The presence of circumferential waves was confirmed by pushing on a tube with one transducer and measuring with the same transducer and another transducer oriented at 90° (FIGS. 3A and 3B). The longitudinal wave that was generated was a bending or asymmetric mode, and the wall motion along the tube in the longitudinal direction for walls 1 and 2 are shown in FIG. 4. In this type of mode, the artery walls move in phase with each other similar to the bending of a beam. The circumferential wave has been observed to be a symmetric mode; that is, the artery walls are 180° out-of-phase with each other as depicted from walls 3 and 4 along the length of the tube in FIG. 5.

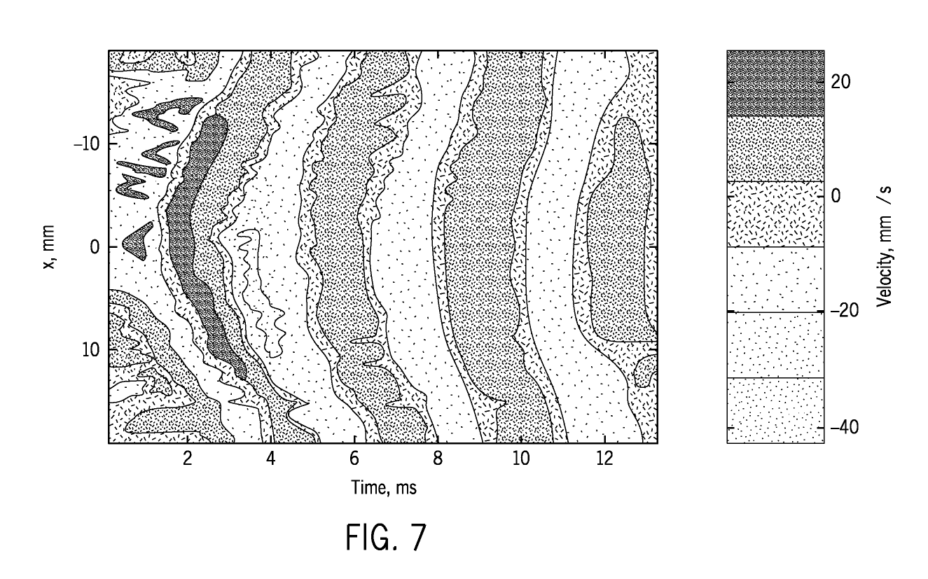

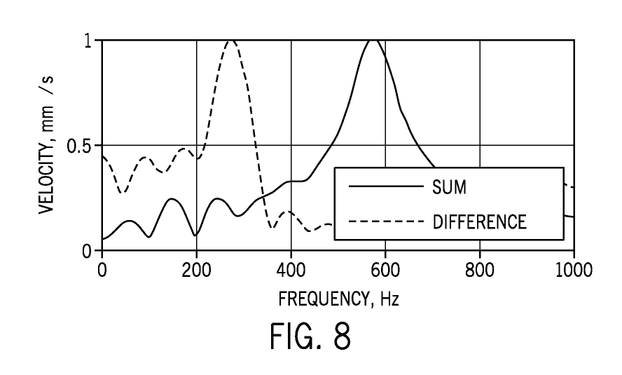

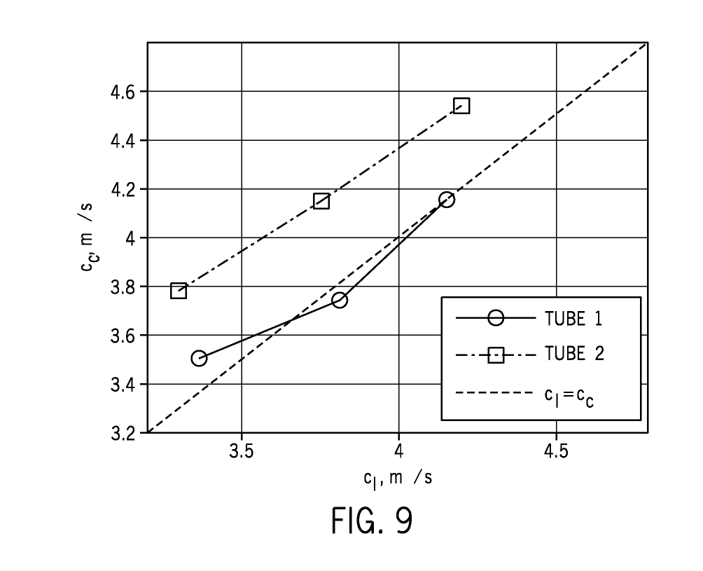

One way to isolate this symmetric mode for analysis is to subtract the wall motion from the two walls measured by a single transducer. This operation cancels motion that is in-phase, i.e., asymmetric motion, and will emphasize the motion that is out-of-phase, symmetric motion. To illustrate this the sum (FIG. 6) and the difference (FIG. 7) of the motion detected from the top and bottom walls of an artery at 40 mmHg were taken, a fast Fourier transform was performed, and a distinct frequency difference was found as shown in FIG. 8. We compared c₁ and c_{c} in the tubes and found very good agreement in tube 1 and a bias in tube 2 (FIG. 9). It was expected that both wave speeds would be similar because the material is isotropic. The c₁ and c_{c} values for the artery shown in FIG. 5 show similar trends but the c_{c} values are lower than the c₁ values. Values of c₁ and c_{c} in tubes and arteries were measured. The speeds measured in isotropic tubes were similar, but were different in the excised artery, which is a direct measure of anisotropy. Some hysteresis was observed in the wave speeds indicating that viscoelasticity is also present.

A method was performed that can extract information about both the longitudinal and circumferential wave speeds in tubes and arteries. It was first observed through a unique experimental design that both the longitudinal and circumferential waves could be induced with a focused radiation force beam. It was also observed that asymmetric motion from the transducer that was the source of the push force and symmetric motion orthogonal to the push transducer. The method of subtracting motion from both walls was used to isolate the symmetric behavior and then Fourier techniques were used to obtain information about the circumferential wave speeds.

The longitudinal and circumferential wave speeds were measured in two custom-made tubes and the speeds were compared assuming isotropicity of the material. The results from the first tube at three different transmural pressures matched very well. The second tube had a linear correlation between the c₁ and c_{c} values but the c_{c} values were biased high.

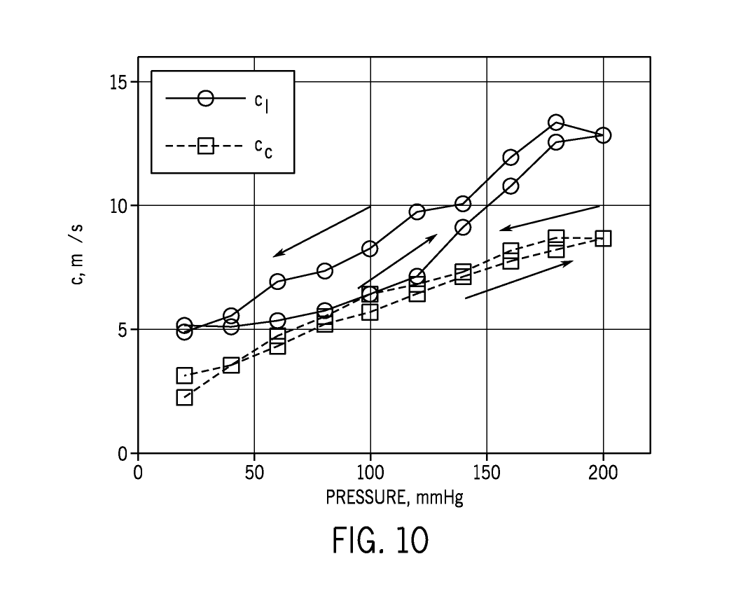

Lastly, the values of c₁ and c_{c} were measured as the vessel was pressurized from 20 to 200 mmHg and then depressurized back to 20 mmHg, and the results are shown in FIG. 10. The values of c₁ were higher than the values of c_{c}, which indicated anisotropy. Also, some hysteresis was present, which is indicative of viscoelasticity of the material.

The present invention has been described in terms of one or more preferred embodiments, and it should be appreciated that many equivalents, alternatives, variations, and modifications, aside from those expressly stated, are possible and within the scope of the invention.

1. A method for measuring and isolating a wave speed within a vessel wall of a substantially cylindrical vessel using an ultrasound imaging system, the vessel having a longitudinal axis and a circumference that is orthogonal to the longitudinal axis, the steps of the method comprising:
(a) applying, with a first transducer of the ultrasound imaging system, ultrasound to a first region of the vessel wall that is proximal to the first ultrasound transducer and to a second region of the vessel wall that is distal to the first ultrasound transducer, wherein the ultrasound induces a longitudinal wave that propagates within the vessel wall along the longitudinal axis and a circumferential wave that propagates within the vessel wall along the circumference;
(b) measuring, using the first transducer, a motion of the vessel wall at a first location on the circumference, the vessel wall experiencing the propagating longitudinal wave along the longitudinal axis and the propagating circumferential wave about the circumference;
(c) measuring, using the first transducer or a second transducer of the ultrasound imaging system, a motion of the vessel wall at a second location on the circumference, the first location and the second location being positioned at different locations on the circumference;
(d) isolating a circumferential wave speed of the propagating circumferential wave by computationally analyzing, using a processor of the ultrasound imaging system, a difference between the motion of the vessel wall at the first location and the motion of the vessel wall at the second location; and
(e) generating a report indicating material properties of the vessel using the circumferential wave speed.

2. The method of claim 1, wherein the ultrasound applied in step (a) is an unfocused ultrasound push beam that is applied to both the first region and the second region.

3. The method of claim 2, wherein the ultrasound applied in step (a) includes at least two unfocused ultrasound push beams each applied at a different lateral location along the longitudinal axis such that each unfocused ultrasound push beam is applied to different first and second regions at the different lateral locations.

4. The method of claim 1, wherein the ultrasound applied in step (a) includes a first focused ultrasound applied to the first region and a second focused ultrasound applied to the second region.

5. The method of claim 4, wherein applying the first focused ultrasound in step (a) includes at least one of focusing or steering a focused ultrasound push beam to the first region, and applying the second focused ultrasound in step (a) includes at least one of focusing or steering the focused ultrasound push beam to the second region.

6. The method of claim 1, wherein the ultrasound applied in step (a) includes a focused ultrasound push beam having a focal region that extends from the first region to the second region, such that the ultrasound is simultaneously applied to the first region and the second region.

7. The method of claim 6, wherein the ultrasound applied in step (a) includes at least two focused ultrasound push beams each applied at a different lateral location along the longitudinal axis such that each focused ultrasound push beam is applied to different first and second regions at the different lateral locations

8. The method of claim 1, wherein the substantially cylindrical vessel is a synthetic tube, a blood vessel, an esophagus, or a section of the gastrointestinal tract.

9. The method of claim 1, wherein computationally analyzing comprises Fourier transforming.

10. The method of claim 1, the method further comprising selecting the first location and second location based on a geometry of the vessel.

11. The method of claim 10, wherein the geometry of the vessel is measured using the ultrasound imaging system.

12. The method of claim 1, the method further comprising isolating a longitudinal wave speed of the propagating longitudinal wave and generating the report using the longitudinal wave speed.

13. The method of claim 12, wherein isolating a longitudinal wave speed comprises using a time of flight based technique, using a Radon transform based technique, or using a Fourier transform based technique.

14. The method of claim 1, wherein the first or second transducer is a linear array transducer, and wherein measuring at the first or second location comprises measuring over a longitudinal section of the vessel wall that includes the first or second location, measuring over a circumferential section of the circumference, or a combination of measuring over a longitudinal section of the vessel wall that includes the first or second location and measuring over a circumferential section of the circumference.

15. The method of claim 1, the method further comprising measuring, using the ultrasound imaging system, a motion of the vessel wall at a third location on the circumference and a motion of the vessel wall at a fourth location on the circumference that is different from the third location.

16. The method of claim 15, wherein the first and second locations are positioned substantially opposite on another on the circumference, and the third and fourth locations are positioned substantially opposite one another on the circumference and at an angle of about 90° relative to the first and second location.

17. The method of claim 15, wherein the motion of the vessel wall at the first location and the motion of the vessel wall at the second location are measured using the first transducer, and wherein the motion of the vessel wall at the third location and the motion of the vessel wall at the fourth location are measured using the second transducer.

18. The method of claim 1, wherein the motion of the vessel wall at the second location on the circumference is measured using the first transducer and step (d) includes computing the difference by subtracting the motion of the vessel wall at the first location and the motion of the vessel wall at the second location to cancel motion in the vessel wall that is in-phase.

19. A method for measuring and isolating a wave speed within a wall of a bodily lumen using an ultrasound imaging system, the bodily lumen having a longitudinal axis and a circumference that is orthogonal to the longitudinal axis, the steps of the method comprising:
(a) measuring, using a first transducer of the ultrasound imaging system, a motion of the wall of the bodily lumen at a first location on the circumference, wherein the motion of the bodily lumen at the first location is caused by a longitudinal wave propagating along the longitudinal axis and a circumferential wave propagating about the circumference, and wherein the longitudinal wave and the circumferential wave are induced in the wall of the bodily lumen by physiological motion intrinsic to the bodily lumen;
(b) measuring, using the first transducer or a second transducer of the ultrasound imaging system, a motion of the wall of the bodily lumen at a second location on the circumference that is different from the first location, wherein the motion at the second location is caused by the longitudinal wave and the circumferential wave induced in the wall of the bodily lumen by the physiological motion intrinsic to the bodily lumen;
(c) isolating a circumferential wave speed of the propagating circumferential wave by computationally analyzing, using a processor of the ultrasound imaging system, a difference between the motion of the wall of the bodily lumen at the first location and the motion of the wall of the bodily lumen at the second location; and
(d) generating a report indicating mechanical properties of the bodily lumen using the circumferential wave speed.

20. The method of claim 19, wherein the bodily lumen is a blood vessel and the physiological motion intrinsic to the bodily lumen is motion caused by pulsatile blood flow through the blood vessel.

21. The method of claim 19, wherein the bodily lumen is an esophagus and the physiological motion intrinsic to the bodily lumen is motion caused by swallowing.

22. The method of claim 19, wherein computationally analyzing comprises Fourier transforming.

23. The method of claim 19, the method further comprising selecting the first location and second location based on a geometry of the bodily lumen.

24. The method of claim 23, wherein the geometry of the bodily lumen is measured using the ultrasound imaging system.

25. The method of claim 19, the method further comprising isolating a longitudinal wave speed of the propagating longitudinal wave and generating the report using the longitudinal wave speed.

26. The method of claim 25, wherein isolating a longitudinal wave speed comprises using a time of flight based technique, using a Radon transform based technique, or using a Fourier transform based technique.

27. The method of claim 19, wherein the motion of the vessel wall at the second location on the circumference is measured using the first transducer and step (c) includes computing the difference by subtracting the motion of the vessel wall at the first location and the motion of the vessel wall at the second location to cancel motion in the vessel wall that is in-phase.
